(19) United States
(12) Patent Application Publication
(10) Pub. No.: US 2017/0189149 A1
(43) Pub. Date: Jul. 6, 2017

(54) ULTRASONIC TOOTH CLEANING APPARATUS AND METHOD

(71) Applicant: TCD Consulting LLC, Henderson, NV (US)

(72) Inventors: Walter Golub, Henderson, NV (US); Phillip Chiappe, Southampton, NY (US); Charles Dresser, Newton, MA (US)

(21) Appl. No.: 15/408,888

(22) Filed: Jan. 18, 2017

Related U.S. Application Data
(63) Continuation-in-part of application No. 14/921,574, filed on Oct. 23, 2015.
(60) Provisional application No. 62/069,305, filed on Oct. 27, 2014.

(51) Int. Cl. A61C 17/20; A61C 1/00; A61C 19/06; A61N 7/00; A61C 1/07

(52) U.S. Cl. CPC A61C 17/20; A61N 7/00; A61C 1/07; A61C 19/066; A61C 1/0069; A61N 2007/0078; A61N 2007/0039

(57) ABSTRACT

The system relates to a device and method for ultrasonic whitening of teeth through cavitation. The device and method provide functions to consistently whiten dental surfaces. Ultrasound is generated and coupled through a removable mouthpiece into a coupling fluid in which teeth and gums are at least partially submerged. Several different embodiments for the system, including variations in the mouthpiece, coupling components, and ultrasonic energy components are disclosed.

### CROSS-REFERENCE TO RELATED APPLICATION

The present application is a Continuation In Part of U.S. patent application Ser. No. 14/921,574, filed Oct. 23, 2015

### FEDERALLY-SPONSORED RESEARCH

None

### BACKGROUND

Good oral hygiene requires that plaque be removed from teeth and gingiva. If plaque is not removed the bacteria that live within plaque will eventually produce enough acid adjacent the enamel to form cavities. Currently toothbrushes, floss and mouthwash are used by many, daily in an attempt to practice good oral hygiene. For many these items work well and may be used without issue. For others, such as: the elderly, handicapped and young children these items are difficult to use and may require the assistance of an aide for the mouth to be cleaned properly.

Toothbrushing and flossing are manual processes that are performed in large part to remove plaque. As they are manual processes, brushing and flossing are prone to being performed inconsistently and ineffectively. Even those physically capable of brushing and flossing without issue may not be removing enough of the plaque on a consistent basis to prevent decay and ensure good oral hygiene. Automating the teeth cleaning process would allow for effective removal of plaque consistently.

Brushing requires the use of toothpaste and a toothbrush. Like any form of polishing, the effect of brushing on teeth is the gradual removal of the outer surface of enamel. Unlike most of the tissue that humans are comprised of, tooth enamel does not regenerate. This means every time one brushes their teeth they are irreversibly removing enamel. Brushing can also damage the gums. Gum recession, a condition that may require surgical treatment, is caused largely by over-brushing. Currently, one should brush enough to prevent decay, but not so much that oral tissue is damaged.

Ultrasonic cleaning technology is currently used for cleaning dental instruments and may be advantageously implemented to address the aforementioned problems associated with the daily cleaning of the mouth. Information relevant to attempts to address these problems can be found in U.S. Pat. Nos. 5,138,733, 7,044,737, 7,269,873, and 8,769,753. However, these references suffer from one or more of the following disadvantages:

Toothpaste and saliva are a poor ultrasonic medium and will result in very poor ultrasound efficiencies. Toothpaste and salvia form gaseous bubbles and foam. Gas bubbles or foam within an ultrasonic medium act to dampen the forces from the ultrasonic waves. Compression forces of the ultrasonic wave compress gas bubbles decreasing their diameter. Tensile forces of the ultrasonic wave likewise cause the gas bubbles to increase in size. Energy is dissipated into the ultrasonic medium as heat as a result of the changes in size of the gas bubbles. The result of having gas bubbles within the ultrasonic medium is poor transmission of ultrasound. The use of toothpaste and salvia as a medium for ultrasound transmission is apt to require greater ultrasonic energy. The use of a toothbrush to deliver ultrasound to clean dental surfaces is therefore impractical, as it requires coupling of ultrasound through a poorly controlled and inefficient medium.

Reflecting ultrasound from a reflection plate to irradiate buccal dental surfaces is inefficient. Ultrasound is more likely to scatter when it is directed at a target that is small relative the wavelength of the ultrasound. Ultrasound wavelengths typically used in cleaning applications are about 28 KHz, thus having a wavelength of about 5 cm in water. Therefore targets in the mouth are apt to be too small to reflect ultrasound efficiently. Poor ultrasonic efficiency requires greater energy input. The use of large high power ultrasonic transducers within the mouth, and losses through a reflection means of directing ultrasound, may result in excess heat generation. Without a means of detecting overheating, the large high powered ultrasonic transducers may pose a safety risk.

A non-removable mouthpiece adds to the cost of a device, as the mouthpiece will not be able to be replaced independent of the entire device. Just as currently mechanical toothbrushes comprise a consumable brush head and a non-consumable body, a removable mouthpiece is desired that will allow for easy replacement.

Ultrasound is not efficiently transmitted by the bristle of a toothbrush. Coupling ultrasound into the mouth through or near a bristle will not introduce the energy needed to result in ultrasonic cleaning through cavitation. This is especially true at higher ultrasonic frequencies as cavitation requires greater energy at higher frequencies. The bristle of a toothbrush is not stiff enough to produce the pressure changes needed in the ultrasonic medium to generate cavitation, which is the primary means of ultrasonic cleaning.

Ultrasonic cleaning applications that clean through cavitation typically use lower ultrasonic frequencies in the range of 20 KHz-100 KHz. This is because the cavitation threshold is reached with less ultrasonic power at these frequencies. The use of high frequency ultrasound is typically used for applications such as acoustic streaming. Using high frequency ultrasound to acoustically stream microbubbles over dental surfaces does not clean through cavitation. Streaming microbubbles over the dental surface does not impart high levels of disruptive forces on the surfaces being cleaned however and is therefore not likely to clean as well means that include cavitation.

Cavitation results in momentary local temperatures and pressures that break up and remove surface debris. The temperatures resulting from the collapse of vapor bubbles forming cavitation are often higher than 1000K. However, because of the short-lived nature of this enormous energy cleaning through cavitation is often gentle to the part being cleaned. Ultrasonic cleaning through cavitation has been shown to remove plaque from dentures in scientific studies and is used daily in industry.

An embodiment of the present device may also be directed to the process of tooth whitening. Typically, a tooth whitening process involves applying a gel or paste containing a percent of peroxide, also called a whitening agent. Whitening agents most commonly include hydrogen peroxide or carbamide peroxide, though other forms of peroxide may be used. The whitening process may involve application of the whitening agent using mouthpiece, where the mouthpiece may be custom made for a user from dental impressions.

For the foregoing reasons, there is a need for a device that is easily operated to automatically and consistently clean the teeth and gums of the user through ultrasonic cavitation. The device may also be used for tooth whitening purposes.

### SUMMARY

In order to improve the ease and consistency of daily cleaning and/or whitening of teeth and gums various embodiments of ways and means of ultrasonic cleaning are realized. Conventional industrial ultrasonic cleaning cleans through cavitation. Cleaning through ultrasonic cavitation is suitable for a fully automatic removal of plaque, biofilm and even tartar present in the mouth. Achieving suitable cleaning by these ultrasonic means in an oral cavity of a user requires that the ultrasound be delivered in a controllable way and that it is directed consistently to all surfaces requiring cleaning in the mouth. This is in order to prevent damage to the mouth or spotty cleaning of the mouth. Additionally the ultrasound must be efficient as excess losses result in high ultrasonic power requirements that are apt to overheat and damage dental tissue or cause discomfort to the user.

A removable mouthpiece may be used in order to reduce the cost of ownership of a device for ultrasonic cleaning. Ultrasound may be coupled efficiently through the mouthpiece and into a coupling fluid that partially fills volumes of the mouthpiece by matching the acoustic impedance of ultrasonic mediums. Cavitation within the coupling fluid may result in cavitation near surfaces of teeth and gums. The temperature of the coupling fluid may be controlled to allow for the most efficient cavitation, and safe operation of ultrasonic cleaning. The cavitation may be directed accurately by locating and orienting ultrasound generating means and controlling a waveform at which the ultrasound is generated at. The waveforms may be modulated in terms of frequency, sweep range, sweep rate, duty cycle, amplitude, pulse time, duty cycle and phase to achieve efficient ultrasonic cleaning.

Accordingly, in one aspect a device for whitening dental tissue includes a mouthpiece being insertable into a mouth of a user. The mouthpiece having at least three sides that partially circumscribe one or more volumes. And, an ultrasound generator that is removably attached to the mouthpiece. Such that, when attached ultrasound generated by the ultrasonic generator is coupled into the volumes through the sides of the mouthpiece.

In some embodiments, a coupling fluid, comprising water, partially fills the volumes, and where the coupling fluid includes a whitening agent.

In some embodiments, a fluid temperature controller is provided that maintains the temperature of the coupling fluid within a desired range. The fluid temperature controller controls the temperature of the coupling fluid by sensing the temperature of the coupling fluid and heating or cooling the coupling fluid.

In some embodiments, the ultrasound generator includes piezoelectric ultrasonic transducers.

In some embodiments, the device includes a battery that powers the ultrasound generator is preferably rechargeable.

In some embodiments, the device additionally directs the generated ultrasound toward interproximal regions of the mouth. In another embodiment, the device directs generated ultrasound to buccal, lingual and occlusal surfaces of the mouth simultaneously.

In some embodiments, the ultrasound generator of the device is operationally responsive to at least one drive signal. The dive signal drives the ultrasound generator about a frequency.

In some embodiments, the ultrasound generator of the device includes more than one ultrasonic transducer. And, the more than one ultrasonic transducer may be driven by independent drive signals. Such that, different ultrasonic transducers may be driven intermittently or at a different phase that other ultrasonic transducers.

In some embodiments, the mouthpiece has contours that parallel the surfaces of the mouth of the specific user using the device. This requires that the mouthpiece be a custom mouthpiece. The forming of the custom mouthpiece may include additive manufacturing processes and/or molding.

In some embodiments, the device includes a handle attached to the ultrasound generator. The handle allows the user to hold onto the device. The handle may optionally include a joint. The joint allows the attitude, or angle at which the user holds the device to be selectable. The handle may optionally be of a ball and socket type, although any hinging means is suitable for the joint.

One aspect of the disclosure relates to a method for whitening and/or cleaning dental tissue. The method for whitening and/or cleaning dental tissue includes inserting a mouthpiece into the mouth of the user. The mouthpiece having one or more volumes partially circumscribed by one or more sides of the mouthpiece, allows one or more teeth of the user fit within the said one or more volumes. Generating ultrasound with an ultrasound generating means. And, coupling the ultrasound through the one or more side of the mouthpiece into the volumes. The ultrasound coupled into the volumes is directed toward buccal, lingual, and occlusal surfaces of the mouth.

In some embodiments, the method for whitening and/or cleaning dental tissue includes filling the volumes partially with a coupling fluid, comprising water.

In some embodiments, the method for whitening and/or cleaning dental tissue includes controlling the temperature of the coupling fluid to be within a desired temperature range. The desired temperature range may be 70-100° F. Controlling the temperature of the coupling fluid may be achieved by heating the coupling fluid or by cooling the coupling fluid.

In some embodiments, the method for whitening and/or cleaning dental tissue includes generating ultrasound through oscillating at least one piezoelectric transducer.

In some embodiments, the method for whitening and/or cleaning dental tissue includes powering the ultrasound generator. Powering the ultrasound generator may be achieved through the use of a battery. Preferably, the battery may be rechargeable.

In some embodiments, the method for whitening and/or cleaning dental tissue includes directing the ultrasound substantially toward one or more interproximal regions of the mouth. In another embodiment, ultrasound is directed significantly toward buccal, lingual, and occlusal surfaces of the mouth simultaneously.

In some embodiments, the method for whitening and/or cleaning dental tissue includes controlling the generation of ultrasound. Controlling the generation of ultrasound may include controlling a center frequency about which ultrasound is generated. A frequency at which the ultrasound is generated may be controlled. Controlling the generation of ultrasound may include sweeping the frequency about the center frequency. A sweep rate that defines the rate at which the frequency is continuously varied about the center frequency may be controlled. Varying the sweep rate may be implemented to prevent damage to dental tissue caused by unwanted resonance. The center frequency may be roughly equal to a natural frequency or a harmonic of one or more ultrasonic transducers used for the generation of ultrasound. The center frequency may be controlled discretely by jumping the value of the center frequency generally between harmonics of said natural frequency.

In some embodiments, the method for whitening and/or cleaning dental tissue comprising generating ultrasound in pulses and controlling a duty cycle. The duty cycle is the proportion of pulse on time to total time. The duty cycle may be controlled and varied continuously or discretely.

In some embodiments, the method for whitening and/or cleaning dental tissue includes forming the mouthpiece to include contours that parallel the mouth of a specific user. Forming the mouthpiece may be performed using additive manufacturing and/or molding processes.

In some embodiments, the method for whitening and/or cleaning dental tissue includes fitting an upper portion of the mouthpiece over an upper arch of the mouth of the user; and fitting a lower portion of the mouthpiece over a lower arch of the mouth of the user.

In some embodiments, the method for whitening and/or cleaning dental tissue includes matching an acoustic impedance of the ultrasound generation means and an acoustic impedance of the mouthpiece. The matching of acoustic impedance of the ultrasound generating means and the mouthpiece may be achieved through an acoustic impedance matching device. Said acoustic impedance matching device may be bonded to the mouthpiece and have an acoustic impedance generally equal to an acoustic impedance of the ultrasound generation means. Matching the acoustic impedance of the ultrasound generating means and the acoustic matching device minimizes the amount of ultrasound that is reflected from an interface between the ultrasound generating means and the acoustic matching device. When the acoustic matching device in bonded to the mouthpiece it is possible to maximize the total amount of ultrasound coupled through the mouthpiece by reducing the amount of ultrasound reflected at a mouthpiece interface.

In some embodiments, the method for whitening and/or cleaning dental tissue includes matching the acoustic impedance of the mouthpiece and an acoustic impedance of the coupling fluid. Matching the acoustic impedance of the mouthpiece and the coupling fluid minimizes the amount of ultrasound that is reflected from an interface between the mouthpiece and the coupling fluid.

In some embodiments, the method for whitening and/or cleaning dental tissue includes holding onto the ultrasound generating means. This may be achieved through the use of a handle.

In some embodiments, the method for whitening and/or cleaning dental tissue includes oscillating more than one ultrasonic transducer. At least one first ultrasonic transducer and at least one second ultrasonic transducer may be operated independently from each other.

In some embodiments, the piezoelectric transducer is comprised of a piezeoelectric stack immediately coupled to a horn-type transducer.

In some embodiments, a horn-type transducer joins with connectors to metal inserts in the mouthpiece.

### BRIEF DESCRIPTION OF THE FIGURES

These and other features, aspects and advantages of the present disclosure will become better understood with regard to the following description, appended claims, and accompanying drawings where:

FIG. 1 depicts a schematic of a version of a device for whitening and/or cleaning teeth and a mouth.

FIG. 2 depicts a schematic of a version of a device for whitening and/or cleaning teeth and a mouth.

FIG. 3 depicts a schematic of a version of an ultrasound generator and mouthpiece.

FIG. 4 depicts a schematic of a version of an ultrasound generator and mouthpiece.

FIG. 5 depicts a schematic of a version of an ultrasound generator and mouthpiece.

FIG. 6 depicts a schematic of another version of a device for whitening and/or cleaning teeth.

FIG. 7 depicts a schematic of another version of a device for whitening and/or cleaning teeth.

FIG. 8 depicts a pictorial view of a custom mouthpiece that has contours that parallel a mouth of a specific user.

FIG. 9 depicts a schematic showing ultrasound being coupled through one or more sides of a mouthpiece.

FIG. 10 depicts a schematic showing ultrasound being coupled through one or more sides of a mouthpiece.

FIG. 11 depicts a schematic showing ultrasound being coupled through one or more sides of a mouthpiece.

FIG. 12 depicts a schematic showing ultrasound being coupled through one or more sides of a mouthpiece.

FIG. 13 depicts graphs of ultrasonic parameters.

FIG. 14 depicts graphs of ultrasonic parameters.

FIG. 15 depicts graphs of ultrasonic parameters.

FIG. 16 depicts graphs of ultrasonic parameters.

FIG. 17 depicts graphs of ultrasonic parameters.

FIG. 18 depicts a flow chart of a controller of ultrasound generation.

FIG. 19 depicts a table with exemplary ranges of ultrasonic parameters.

FIG. 20 depicts a schematic of a version of a device for whitening and/or cleaning teeth and a mouth, which includes a horn-type transducer

FIG. 21 depicts a schematic-cross section of a version of a device for whitening and/or cleaning teeth and a mouth, which includes a horn-type transducer

FIG. 22 depicts a version of a device which includes a waveguide

### DETAILED DESCRIPTION

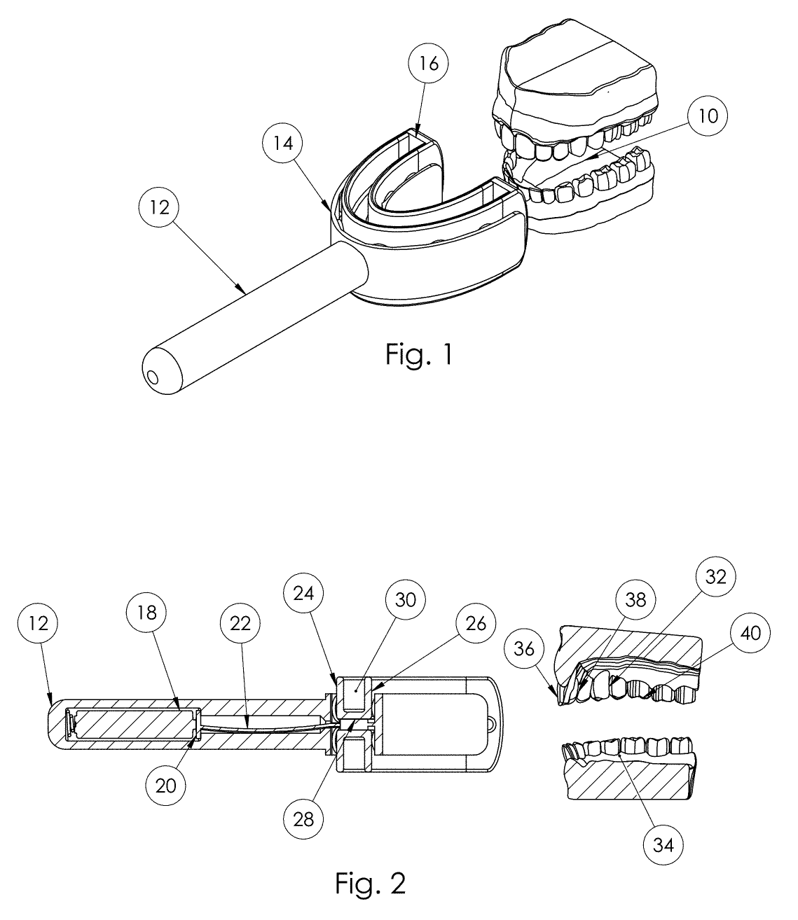

Referring to FIG. 1, a version of the present disclosure, a device for whitening and/or cleaning a mouth of a user, is shown with: a mouth 10. Herein, the terms “cleaning” and whitening” may be used interchangeably. Herein, the term “dental tissues” may refer to any of the hard dental tissues of enamel, dentin and cementum. The version illustrated in FIG. 1 is shown having a handle 12 that is attached to an ultrasound generator 14. The handle 12 allows the user to hold onto the device when it is inserted within the user's mouth 10. While an elongated, tubular-type handle is shown, differently shaped handles may also be used, with any such handle preferably housing the electronic components described below. Surrounding the ultrasound generator 14 is a mouthpiece 16. The mouthpiece 16 is removable from the ultrasound generator 14.

FIG. 2 shows a cross-section of the device in FIG. 1. Within the handle 12; a battery 18, an ultrasonic controller 20, and one or more electrical connections 22 are housed. The battery 18 provides a source of electrical power, powering the ultrasonic controller 20 and the ultrasound generator 14. One or more drive signals are provided by the ultrasonic controller 20 by way of the one or more electrical connections 22 to the ultrasound generator 14. The ultrasound generator operates, generating ultrasound, in response to these one or more drive signals. The ultrasonic controller 20 therefore controls the generation of ultrasound. The cross-section in FIG. 2 shows at least three sides that make up an upper half of the mouthpiece. An upper buccal side 24, an upper lingual side 26, and an upper occlusal side 28 together partially circumscribe an upper mouthpiece volume 30. Inserting the mouthpiece into the mouth of the user results in teeth belonging to an upper arch 32 being positioned within the upper mouthpiece volume 30, such that: Upper buccal surfaces 36 are adjacent the upper buccal side of the mouthpiece 24. Upper lingual surfaces 38 are adjacent the upper lingual side of the mouthpiece 26. And, Upper occlusal surfaces 40 are adjacent the upper occlusal side of the mouthpiece 28. A lower half of the mouthpiece is illustrated in FIG. 2 as being a mirror image of the upper half of the mouthpiece. A lower arch 34 of the user will therefore fit within a lower mouthpiece volume during insertion. Ultrasound generated by the ultrasound generator is coupled through one or more of the sides of the mouthpiece and into the upper and lower mouthpiece volumes.

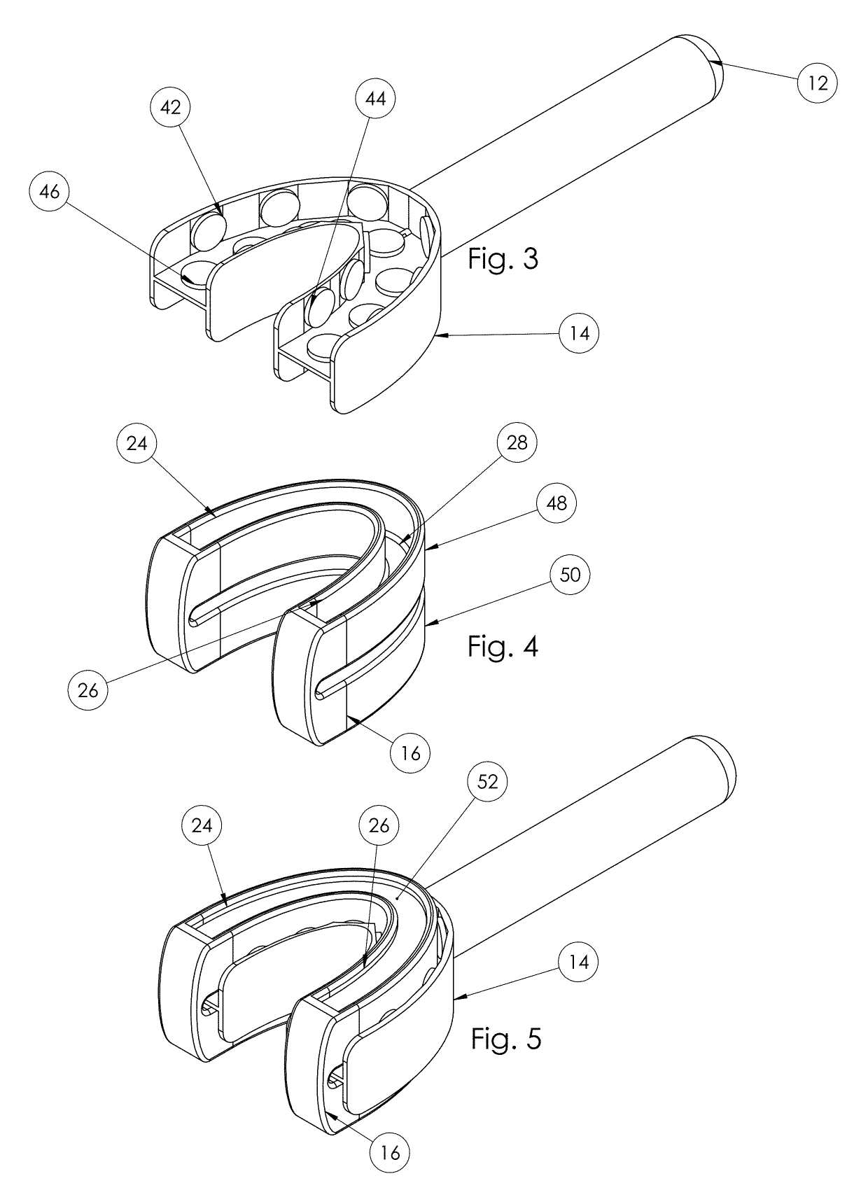

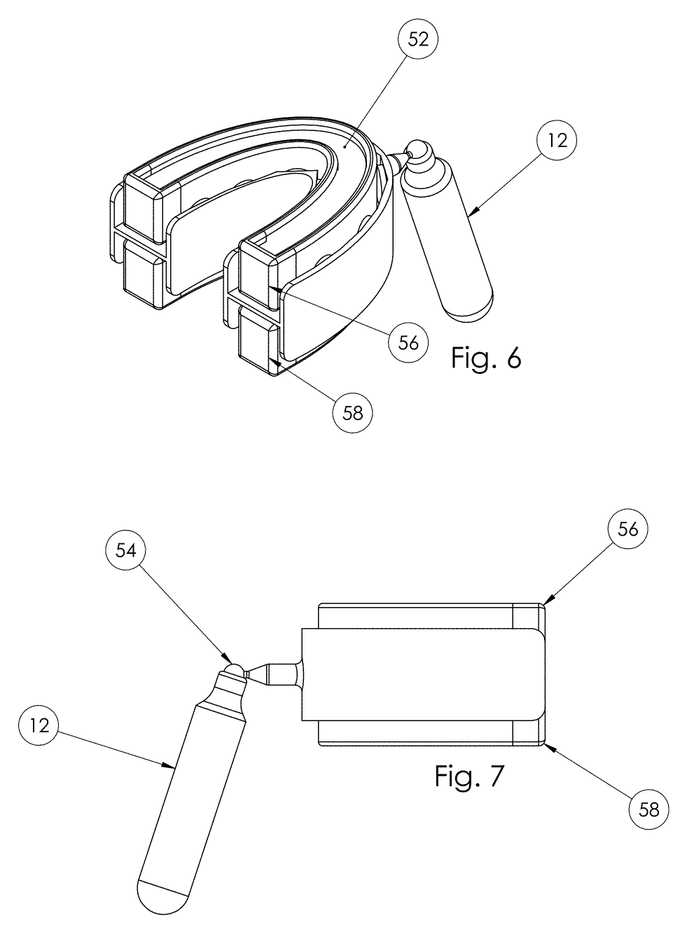

FIGS. 3-5 shows an exploded view of a version of the present disclosure. In the version of the present disclosure shown in FIG. 3, the ultrasound generator comprises a multitude of ultrasonic transducers, which are located such that during insertion of the device they are within the mouth of the user. An upper half of the ultrasound generator may comprise: one or more upper buccal ultrasonic transducers 42, upper lingual ultrasonic transducers 44, and upper occlusal ultrasonic transducers 46. A lower half of the ultrasound generator is illustrated in FIG. 3 as being a mirror image of the upper half of the ultrasound generator. The ultrasonic transducers generate ultrasound by producing a series of pressure changes in an ultrasonic medium. The pressure changes are typically generated through a small change in volume of the ultrasonic medium. The small changes in volume of the ultrasonic medium are the result of a change in at least one dimension of the ultrasonic transducer. The size of the change in at least one dimension of the ultrasonic transducer relates to an amplitude of the pressure changes and an amplitude of the generated ultrasound. The ultrasonic transducers being driven by one or more drive signals oscillate in said dimension at a frequency that is above 20 KHz and below 10 MHz. The ultrasonic transducers may be piezoelectric transducers. Piezoelectric transducers that are lead-free are advantageously selected for use in applications within the mouth. PIC700 ultrasonic transducer material from PI Ceramic GmbH of Lindenstrasse, Germany is lead-free and comprises bismuth-sodium-titanate. Of course, other suitable transducer materials may also be used. The ultrasonic transducers shown in FIG. 3 are shown as disks being defined by a diameter and a thickness.

FIG. 4 shows a version of the mouthpiece. The mouthpiece shown in FIG. 4 is one piece, having an upper half 48 and a lower half 50. The mouthpiece may alternatively be comprised of two separate and distinct elements or, an upper portion and a lower portion. The mouthpiece 16 approximately matches the acoustic impedance of the ultrasound generator 14 and a coupling fluid that at least partially fills the one or more volumes of the mouthpiece 30. The mouthpiece may comprise: rubber, silicone, PVC, foam, or any elastomeric polymer. The coupling fluid may include but are not limited to one or more of the following ultrasonic mediums: water, glycerin, propylene glycol, calcium hydroxide, carbapol, one or more stabilizing agents, one or more solvents, one or more flavor additives, or one or more surfactants. Stabilizing agents act to increase the viscosity of the coupling fluid. An increased viscosity allows the coupling fluid to be handled and applied more easily as a paste or gel into the one or more volumes of the mouthpiece. Suitable stabilizing agents include but are not limited to: gelling agents, thixotropic additives, and gums. The presence of solvents in the coupling fluid would act to chemically break down biofilm and/or inhibit bacteria on the dental surfaces. Suitable solvents may include but are not limited to: Alcohol, chlorhexidine gluconate, cetylpyridinium chloride hexetidine, benzoic acid, methyl salicylate, triclosan, benzalkonium chloride, methylparaben, hydrogen peroxide, domiphen bromide, fluoride, enzymes, calcium, essential oils, such as: phenol, thymol, eugenol, eucalyptol, menthol. Flavor additives may be used in the coupling fluid to improve the taste and appeal for using the device. Suitable flavor additives include but are not limited to: sorbitol, sucralose, stevia, sodium saccharin and xylitol, which additionally acts to inhibit acid causing bacteria. The presence of surfactants in the coupling fluid acts to lower the surface tension of the fluid and increases ultrasonic cavitation. Suitable surfactants include but are limited to: detergents, wetting agents, emulsifiers, and dispersants. Microbubbles or, ultrasound contrast mediums, such as: SonoVue, Optison or Levovist, may also be added to the coupling fluid.

An embodiment of the present device may also be directed to the process of tooth whitening. One embodiment of the present device may be used with whitening agents, where one or more of the coupling fluid, solvents, or surfactants contain a percentage of peroxide, where hydrogen peroxide and carbamide peroxide are preferred. The device's ultrasonic features may be used with whitening agents, using similar or different operating parameters as are used for ultrasonic cleaning processes that do not include whitening agents. Preferably, for tooth whitening purposes, a tooth-whitening agent containing peroxide, preferably hydrogen peroxide or carbamide peroxide, is included as percentage of the coupling fluid.

FIG. 5 shows version of the present disclosure illustrated in FIG. 3 with the mouthpiece 16 attached to the ultrasound generator 14. With the mouthpiece attached to the ultrasound generator 14 and the volumes 30 filled with coupling fluid 52, the generated ultrasound is coupled through the buccal 24, lingual 26, and occlusal 28 sides of the mouthpiece into the coupling fluid 52.

Ultrasound when introduced into the coupling fluid at sufficient levels produces cavitation. Cavitation occurs when pressure waves, produced by ultrasound, generate low pressure regions within the coupling fluid forming a multitude of vapor bubbles. When the pressure waves are inverted the vapor bubbles collapse as the formally low pressure regions experience high pressure. The collapse of these vapor bubbles creates localized temperatures that have been measured as high as 5000K and localized pressures that have been measured as high as 1000 atm. These high localized temperatures and pressures that exist only momentarily remove particulate from dental tissue that is adjacent to the cavitation. Providing cavitation adjacent to teeth and gum surfaces within the mouth breaks up and removes plaque.

The efficiency of ultrasonic cleaning is a function of the temperature of the coupling fluid. An elevated temperature allows cavitation to be achieved at low pressures regions that have slightly higher pressures than when the coupling fluid is at lower temperatures. This therefore, causes low pressure regions to form more cavitation. For an intraoral application the temperature must be limited in order to prevent dental tissue damage. It is therefore ideal that the temperature of the coupling fluid be controlled at an elevated temperature, in order to maximize cavitation, that is below a threshold level that would cause discomfort or damage to the user. Some versions of the present disclosure may include a fluid temperature controller that is thermosensitively responsive to the temperature of the coupling fluid. Such that, the temperature of the coupling fluid is sensed and the coupling fluid is heated or cooled in order to maintain a coupling fluid temperature within a desired temperature range. The sensing of the coupling fluid temperature may be done thermostatically or through thermoelectric means, such as a thermistor or thermocouple. The fluid temperature controller determines a heating or cooling load required to maintain the coupling fluid temperature within the desired coupling fluid temperature range. Determination of the heating or cooling loads may be achieved through P.I.D., or other equivalent control logic algorithms, by the temperature controller. The heating load may be provided for by controlled heating of the coupling fluid through electric resistance heating, heat generated through oscillation of ultrasonic transducers, or through a thermoelectric heat pump. The cooling load may be provided for by controlled cooling of the coupling fluid through a thermoelectric heat pump, or other equivalent means. Controlled cooling of the coupling fluid additionally reduces the risk of overheating and damaging the dental tissue of the user. The desired temperature range is typically within 70-100° F. and is preferably held within a smaller range that is elevated, but still comfortable to the user, such as 80-90° F.

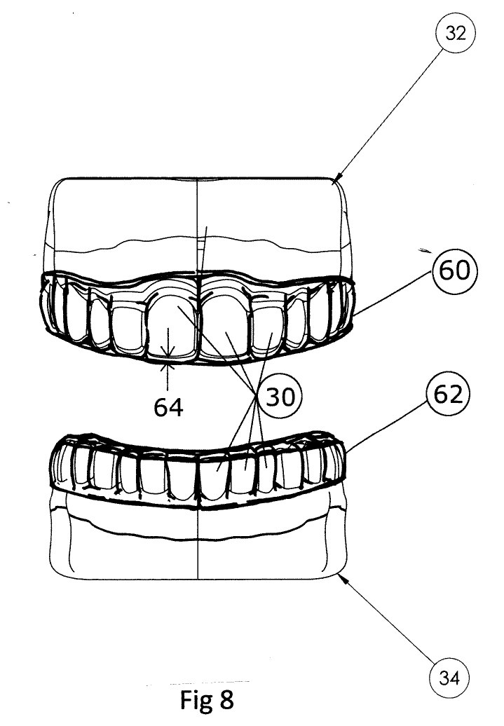

Another version of the present disclosure is shown in FIGS. 7 and 8. The handle 12 is shown having a joint 54. The joint allows for the attitude of the handle relative the mouthpiece to be selected by the user. This allows the user to hold the handle at any number of angles relative the mouthpiece. The joint 54 shown in FIG. 7 is a ball and socket joint although any equivalent hinging mechanism may be used. FIG. 7 also, shows a version of the present disclosure that includes a mouthpiece with an upper portion 56 and a lower portion 58 that is separate and distinct from the upper portion. The upper arch of the user is fitted within the upper portion and the lower arch of the user is fitted within the lower portion. A version of the mouthpiece is also conceived of that includes only a single portion, which houses only one arch of the user at a time. This version of the disclosure has the single portion fitted over a first arch of the user. The device ultrasonically cleans the first arch. Then, the single portion is removed from the first arch and fitted over a second arch of the user. And, the device ultrasonically cleans the second arch. The version of the disclosure with the single portion mouthpiece roughly doubles the amount of time needed to clean the mouth, and halves the number of components in the device.

Referring to FIG. 8 a version of the present disclosure includes a custom mouthpiece. The custom mouthpiece is shown having an upper custom portion 60 and a lower custom portion 62. The custom mouthpiece has contours that parallel the dental surfaces of the user. The contours of the custom mouthpiece allow the custom mouthpiece to fit closely over the upper arch 32 and lower arch 34 of the user. Inserting the custom mouthpiece into the mouth of the user may form multiple volumes 30 that may partially circumscribe one or more individual teeth. The contours of the custom mouthpiece may form a gap 64 between dental surfaces and the mouthpiece. The gap 64 may be of uniform width. Selecting the size of the gap 64 may allow for optimum performance of ultrasonic cleaning. This is because the distance the ultrasound is transmitted in the coupling fluid prior to reaching the dental surfaces is a function of the gap. Minimizing the gap 64 will generally reduce the ultrasonic loses within the coupling fluid and unintended heating of the coupling fluid.

The contours of the custom mouthpiece are to be specially formed for a specific user. The specific user may have a scan or an impression taken of his mouth. The scan or the impression is then used to form the contours of the custom mouthpiece. Forming of the custom mouthpiece may be achieved from the scan of the mouth through additive manufacturing processes, such as: SLA or an equivalent 3-D printing/fabrication technology. A positive of the scan may be printed, through additive manufacturing, and used to generate a mold to form the custom mouthpiece with. Likewise the custom mouthpiece may be formed from a mold made from the impression of the mouth. The contours of the custom mouthpiece may also be formed by inserting a pliable mouthpiece into the mouth of the user and using the mouth of the user directly to form the contours.

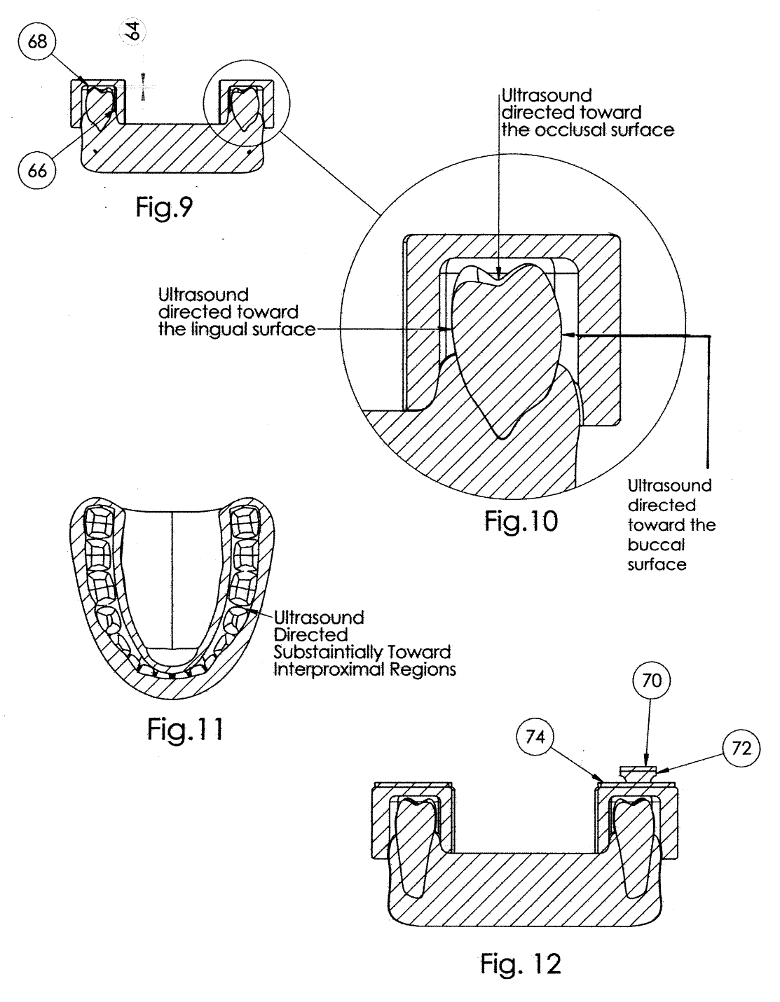

FIG. 9 through 12 show the lower arch of the user with a version of the lower portion of the mouthpiece. FIG. 9 shows a cross-sectional view. A lower lingual side of the mouthpiece 66 rests on the bottom of the mouth. The distance in height of the lower lingual side is greater than the height of teeth in the arch. This difference in distance produces the gap 64 between the occlusal surfaces of the teeth and a lower occlusal side of the mouthpiece 68. The distance of the gap between sides of the mouthpiece and surfaces of the mouth of the user is a parameter that affects the distance that the ultrasound is transmitted through the coupling fluid to reach the surfaces of the mouth to be cleaned.

FIG. 10 shows ultrasound being coupled through the mouthpiece and being directed toward three different surfaces of the mouth; specifically a lower lingual surface, a lower occlusal surface, and a lower buccal surface. In a version of the present disclosure the ultrasonic transducers are independently controlled. In this case, the ultrasound directed at the three different surfaces may have different phases. For example, the ultrasound directed toward the lingual surface may be 180° out of phase of the ultrasound directed toward the buccal surface. And, the ultrasound directed toward the occlusal surface may be 90° out of phase of both the ultrasound directed toward the lingual surface and the ultrasound directed toward the buccal surface. The ultrasound directed toward the buccal surface is generally opposing the ultrasound directed toward the lingual surface. This will result in destructive interference and ultrasonic loses if they are of a same frequency and of a same phase. Having the ultrasound directed toward the lingual surface and the ultrasound directed toward the buccal surface 180° out of phase from one another results in constructive interference and greater ultrasound efficiencies. The ultrasound may be generated by one or more first ultrasonic transducers driven to oscillate at a first frequency and one or more second ultrasonic transducers driven to oscillate at a second frequency. The first frequency may be out of phase of the second frequency. The use of drive signals to independently drive ultrasonic transducers also allows ultrasound to be directed to less than all of the surfaces of the mouth at a time by oscillating the ultrasonic transducers intermittently.

FIG. 11 shows a schematic of a version of the present disclosure having ultrasound that is directed substantially toward one or more interproximal regions. Interproximal regions are some of the more difficult surfaces of the mouth to clean. Directing ultrasound toward interproximal regions results in cavitation occurring adjacent interproximal regions, or in between teeth. Directing the ultrasound may be achieved through positioning and orienting the ultrasonic transducers, or horns or waveguides to direct ultrasound to the desired surfaces.

FIG. 12 shows a cross-sectioned schematic view of a version of the present disclosure. One or more piezoelectric transducers 70 are mated to one or more horns 72. The use of the horn, or waveguide, magnifies the amplitude of the oscillations produced by the mating ultrasonic transducer. The horn 72 is mated to an acoustic impedance matching device 74. The acoustic impedance matching device may comprise: graphite, metal or a dielectric material. Ultrasound will reflect where it is being coupled between two mediums of different impedances. The acoustic impedance matching device matches the impedance of horn or ultrasonic transducer and the mouthpiece allowing for more efficient coupling into and through the mouthpiece.

The mouthpiece may be comprised of a number of materials comprising: rubber, silicone, PVC, elastomeric polymers and foam. The material of the mouthpiece is preferably compliant enough to form a seal around gums in the mouth. The seal prevents leaking of the coupling fluid. Acoustic impedance of the material is a function of the density of the material as well as the acoustic velocity of the material. The material for the mouthpiece ideally matches one or both of the ultrasonic generator or the coupling fluid, thus preventing reflections at a mouthpiece interface. The acoustic impedance matching device may be bonded to the mouthpiece and have an acoustic impedance that generally matches that of the ultrasound generated, ultrasonic transducer, piezoelectric transducer, or horn. And, the mouthpiece may have an acoustic impedance that generally matches that of the coupling fluid.

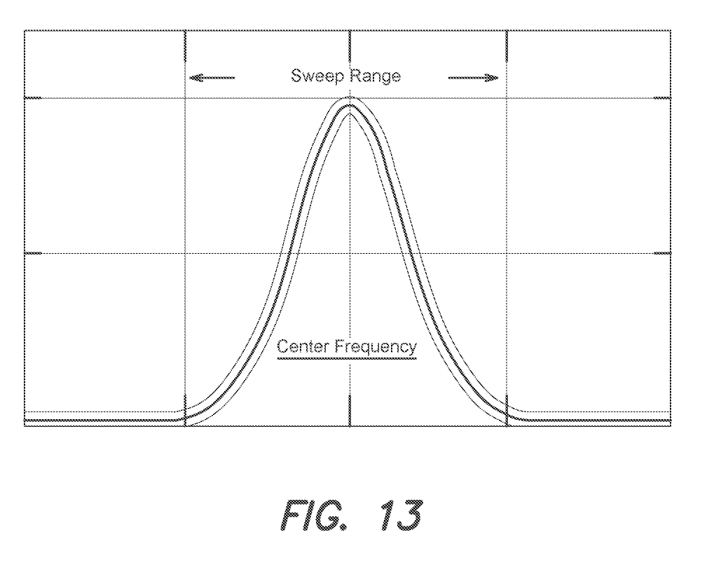

FIGS. 13 through 17 show graphs related to controlling and generating ultrasound. Ultrasonic transducers are oscillated at a frequency above 20 KHz to produce ultrasound. Typically ultrasonic transducers have a natural frequency or resonant frequency based upon their composition and dimensions that at which they oscillate most efficiently. Resulting from manufacturing dimension tolerances an individual ultrasonic transducer will likely have a natural frequency that is not exactly equal to its nominal natural frequency. The result is that the individual ultrasonic transducer will oscillate most efficiently at a frequency that is not equal to that of its nominal natural frequency. In a version of the present disclosure the ultrasound generator and ultrasonic transducers are operationally responsive to one or more drive signals being controlled by an ultrasonic controller. The ultrasonic controller thereby drives the ultrasonic transducers to oscillate at one or more frequencies. Sweeping the frequency the ultrasonic transducers oscillate about a center frequency that is about the same as the nominal natural frequency or a harmonic thereof ensures that the individual ultrasonic transducers are occasionally driven at its actual resonant frequency. The harmonic of the nominal natural frequency is generally defined as being of a frequency that is evenly divisible by a common denominator of the nominal natural frequency. A sweep range determines the variance in frequency that the ultrasonic transducers will oscillate at. FIG. 13 shows a normal distribution that shows frequency in the domain and time or probability in the range.

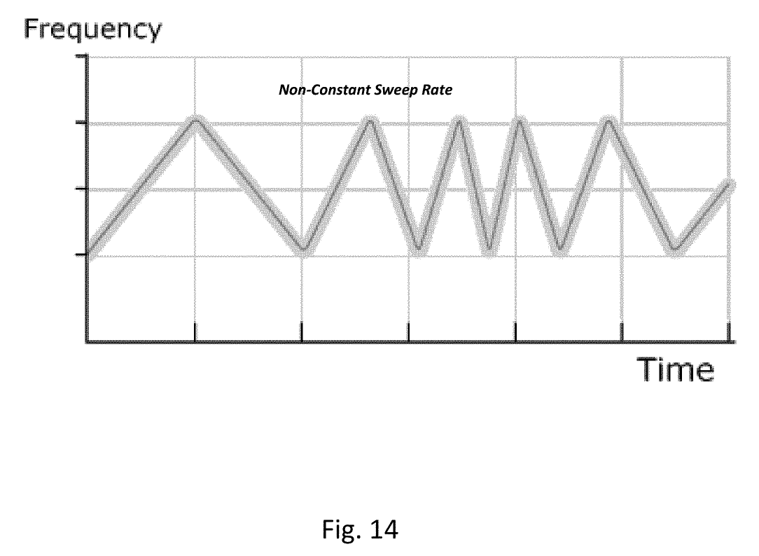

FIG. 14 shows a graph of sweep range vs. time for a version of the present disclosure. It is beneficial to vary or modulate the sweep rate such that it is non-constant. This is because a constant sweep rate would produce a peak amplitude of ultrasound at the resonant point, and periodically generating a peak amplitude of ultrasound would potentially result in harmful resonance.

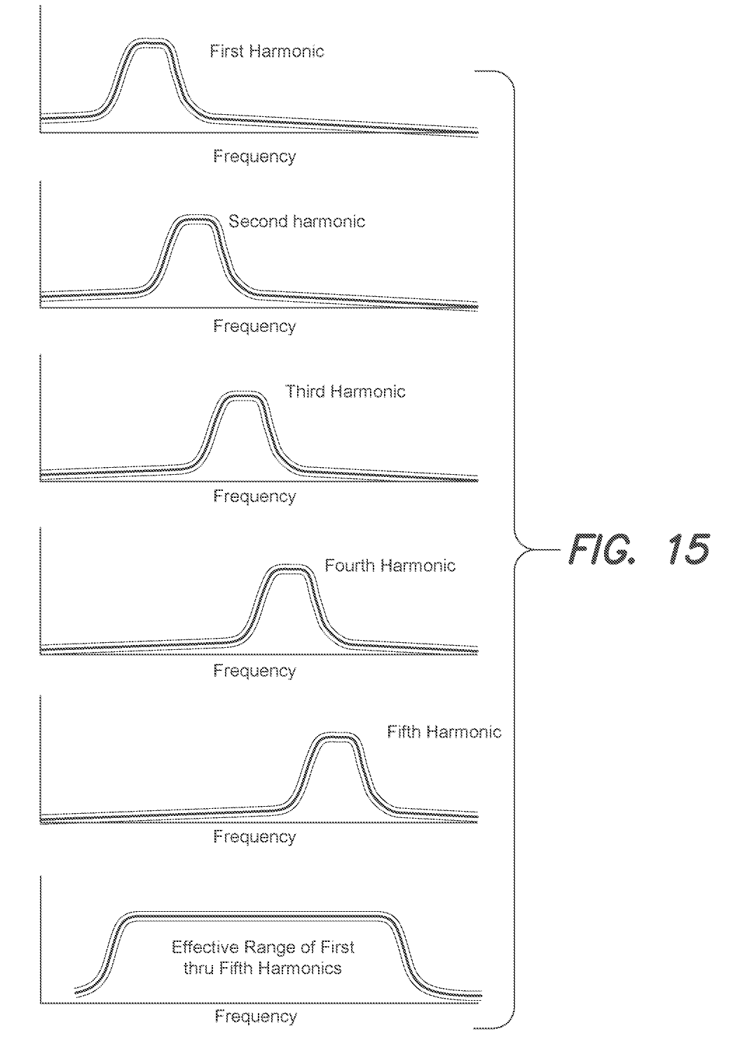

FIG. 15 is a graph showing the size of cavitation producing vapor bubbles. Different frequencies of ultrasound are better suited for cleaning particles of different sizes. Large particles are typically removed best with low ultrasound frequencies, which produce larger vapor bubbles. Smaller particles are typically removed best with higher ultrasound frequencies, which produce smaller vapor bubbles. As it can be seen the ultrasonic transducers may be oscillated about a harmonic of their natural frequency. Jumping the center frequency that the ultrasonic transducer is oscillating about from one harmonic to another allows for the ultrasound to produce cavitation from different sized vapor bubbles and may achieve efficiently the removal of different size particles. Jumping from one center frequency to another may be performed discretely.

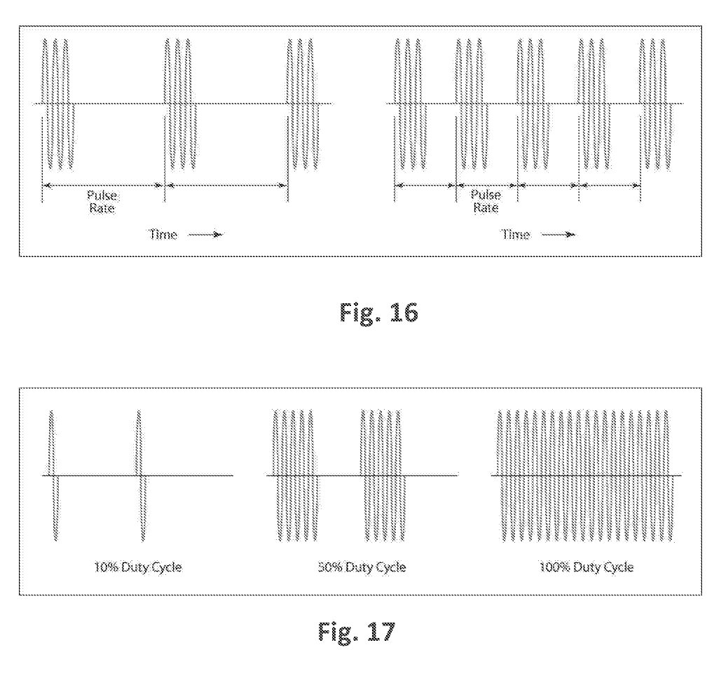

Power of the generated ultrasound is controlled in versions of the present disclosure. An amplitude of the generated ultrasound is a parameter that may be varied to modulate the power of the ultrasound. The greater the amplitude of the ultrasound the more cavitation will occur in the coupling fluid. Another controllable parameter that affects the power of the generated ultrasound is an ultrasound pulse, shown in FIG. 16. Generating the ultrasound in pulses allows the ultrasound to generate less power while maintaining its level of cleaning efficiency. This is ideal for cleaning within the mouth as excessive ultrasound may damage tissue within the mouth. In a version of the present disclosure the ultrasound generator generates the ultrasound in pulses. FIG. 17 shows ultrasound that is generated in pulses having different duty cycles. Varying the duty cycle of the ultrasound provides for varied power levels and may be performed continuously or discretely. The duty cycle may be varied from 0-100%. A degassing step may be performed to remove gas bubbles from the coupling fluid. Typically degassing requires longer pulses, typically in the range 0.5-2 s and low duty cycle, typically in the range of 5-60%. The degassing step loosens gas bubbles from the coupling fluid with the ultrasound and then allows time a delay time in between ultrasonic generation for gas bubbles to escape the coupling fluid. Degassing the coupling fluid is advantageously performed prior to a cleaning step that involves generating ultrasound for cleaning through cavitation. This is because gas bubbles within the coupling fluid contribute to losses in ultrasound. A version of the present disclosure comprises a degassing step.

Ultrasound generation is performed with different parameters for cleaning. Ultrasound generated at frequencies below 500 KHz has a lower cavitation threshold and is better suited for cleaning through cavitation, for this reason less power is needed for cavitation to occur. When ultrasound is generated and the cavitation threshold is not reached acoustic streaming may occur. Acoustic streaming is the movement of the coupling fluid or the generation of standing waves. Acoustic streaming requires parameters other than those used for cleaning through cavitation. Generally higher frequency ultrasound in excess of 500 KHz, and preferably in excess of 1 MHz, is best suited for acoustic streaming as high ultrasound amplitudes may be employed without causing cavitation. Acoustic streaming will produce a flow within the coupling fluid. A flow within the coupling fluid is advantageously implemented in some versions of the present disclosure in order to dislodge and move debris from surfaces of the mouth.

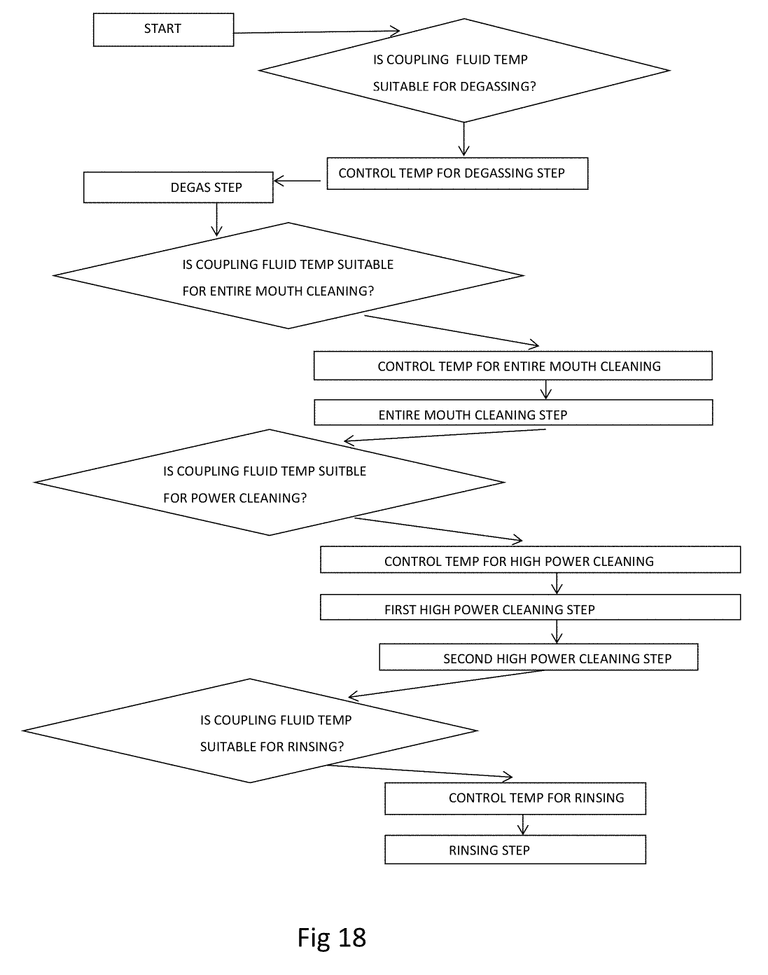

FIG. 18 shows a flow chart for an ultrasonic cleaning process as controlled by the ultrasonic controller in a version of the disclosure. Before initiating the ultrasonic cleaning process, the user fills the volumes of the mouthpiece with coupling fluid and inserts the mouthpiece in his mouth.

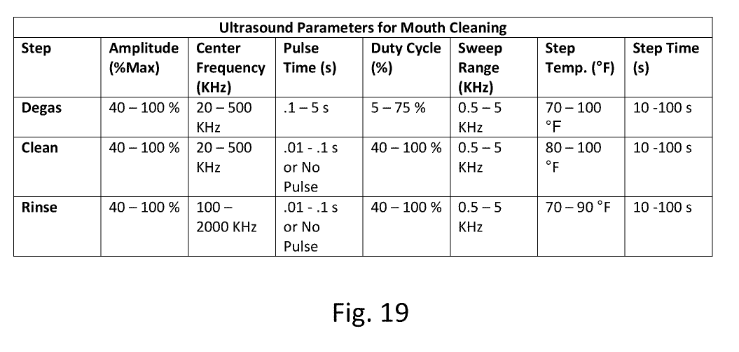

Once the cleaning process is initiated the fluid temperature controller ensures that the coupling fluid is within a desired temperature range for degassing. FIG. 19 shows a table that has preferential ranges for ultrasound parameters used during the cleaning process in a version of the present disclosure. FIG. 19 is not intended to be limiting the scope of the present disclosure. Once the coupling fluid is within a temperature range for degassing, a degas step is performed. The degas step removes air entrained in the coupling fluid by generating ultrasound in pulses with relatively low duty cycles.

Upon completion of the degas step, the fluid temperature controller controls the temperature of the coupling fluid to be within a desired temperature range for entire mouth cleaning. An entire mouth cleaning step is then performed. The entire mouth cleaning step is intended to provide ultrasound to every surface of the mouth simultaneously at a low enough power such that mouth tissue is not damaged. In some versions of the present disclosure ultrasound is directed toward interproximal regions of the mouth and ultrasound generation includes phase modulation.

Upon completion of the entire mouth cleaning step the fluid temperature controller controls the temperature of the coupling fluid to be within a desire temperature range for high power cleaning. A first high power cleaning step directs ultrasound, at high power levels, but only toward some surfaces of the mouth; for example the upper buccal and occlusal surfaces and the lower lingual surfaces. The ultrasound generator may comprise ultrasonic transducers that are driven independently by drive signals from the ultrasonic controller. Thus directing of ultrasound only toward some surfaces of the mouth by operating only some of the ultrasonic transducers.

Upon completion of the first high power cleaning step, a second high power cleaning step is initiated. The second high power cleaning step directs ultrasound only toward those surfaces of the mouth not having ultrasound directed toward them during the first high power cleaning step.

Upon completion of the second high power cleaning step the fluid temperature controller controls the temperature for the coupling fluid to be within a desired temperature range for rinsing. The desired temperature range for rinsing is typically low, because the goal of rinsing is to perform acoustic streaming, not cleaning through cavitation. A rinsing step is intended to rinse debris, which has been removed in previous cleaning steps from the mouth surfaces. Upon completion of the rinsing step, the user removes the mouthpiece from his mouth and the cleaning process is complete. For example, the process above and illustrated in FIG. 18 is provided for example and other processes are possible that include additional or fewer steps, or steps in a different order. For example, the rinsing step may be performed prior to the entire mouth cleaning step. Or, the first and second high power cleaning steps may be omitted.

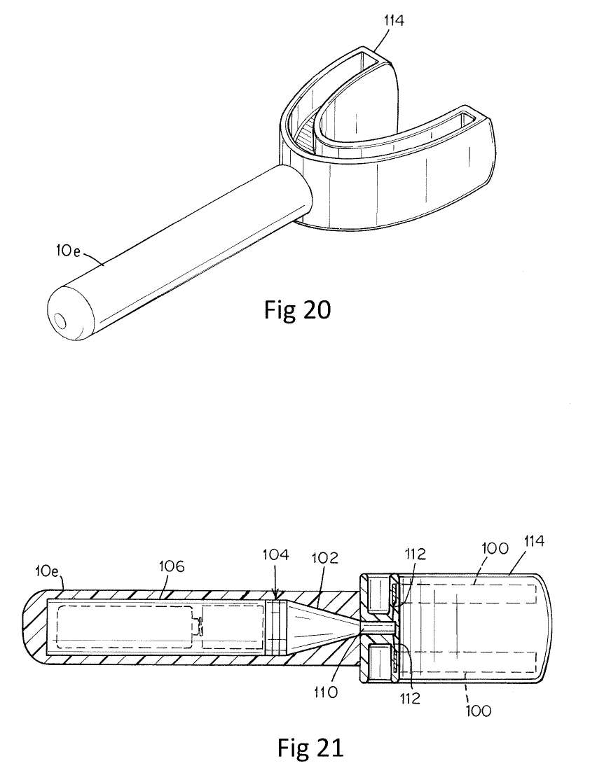

FIG. 20 and FIG. 21 show a version of the present disclosure. Handle housing 10e contains components including component housing 106, piezoelectric stack 104, and horn transducer 102. The hollow tip of horn transducer 102 joins with tip 100 to connectors 112. Preferrably, tip 102 is of a tubular configuration to best transmit ultrasonic energy. Connectors 112 join with metal inserts 112 imbedded in mouthpiece 14.

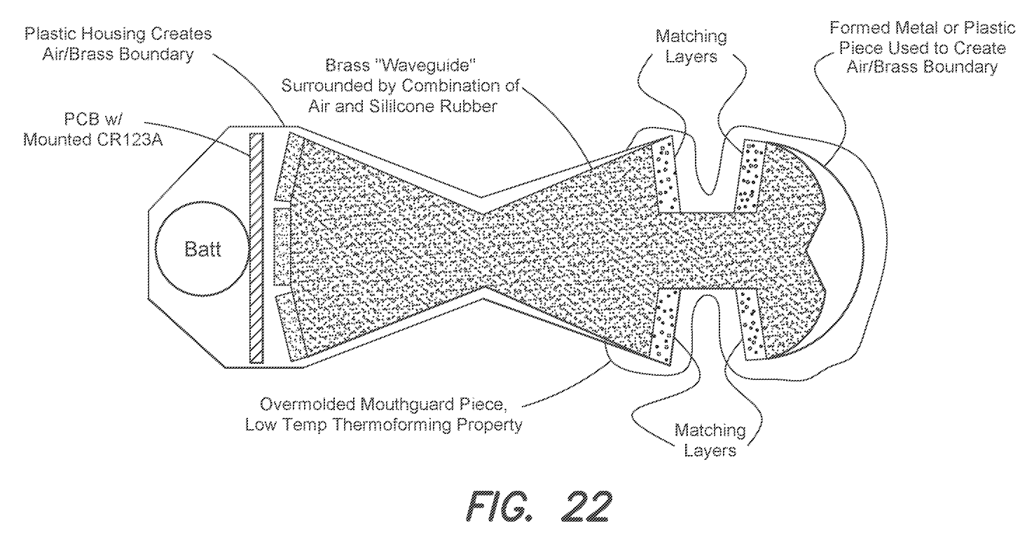

FIG. 22 shows a version of the present disclosure. This embodiment uses a waveguide as shown, preferably made of brass. The waveguide is preferably surrounded by a combination of air and silicone rubber, formed in part with a plastic housing. Components for ultrasound energy generation and control, similar to other embodiments, are also contained in the plastic housing. The waveguide extends to join a mouthpiece as in the above embodiments, including a portion preferably made of plastic or metal that creates a boundary with the air/brass portion.

Although the present disclosure has been described in considerable detail with reference to certain preferred versions thereof, other versions are possible. For example, a means of filling the volumes with the coupling fluid, such as a pump may be incorporated. The ultrasonic transducers may be located partially or totally outside of the mouth and ultrasound is coupled to the mouthpiece through the use of a waveguide or horn. Mouthpieces may come in various sizes for children as well as adults. Therefore, in the spirit and scope of the appended claims should not be limited to the description of the preferred versions contained herein.

1. A device for whitening dental tissue, the device comprising:
a mouthpiece being insertable into a mouth of a user and having at least three sides, with one or more volumes that are partially circumscribed by the sides of the mouthpiece;
and an ultrasound generator removably attached to the mouthpiece that when attached couples ultrasound into the volumes through the sides of the mouthpiece.

2. The device in claim 1 that further includes a coupling fluid at least partially filling the volumes, and where the coupling fluid includes a whitening agent.

3. The device in claim 2, wherein the coupling fluid comprises water.

4. The device in claim 2, wherein the coupling fluid comprises glycerin.

5. The device in claim 2, wherein the coupling fluid comprises propylene glycol.

6. The device in claim 2, wherein the coupling fluid comprises calcium hydroxide.

7. The device in claim 2, wherein the coupling fluid comprises carbapol.

8. The device in claim 2, wherein the whitening agent is at least one of hydrogen peroxide and carbamide peroxide.

9. The device in claim 2, further comprising a fluid temperature controller that controls the temperature of the coupling fluid in the range of 70-100° F. and is thermosensitively responsive to said coupling fluid.

10. The device in claim 2, wherein the coupling fluid includes a stabilizing agent.

11. The device in claim 10, wherein the stabilizing agent is selected from a group consisting of gelling agent, thixotropic additives, and gum.

12. The device according to claim 1, further comprising a handle attached to the ultrasound generator, such that the handle allows the user to hold onto the device.

13. The device in claim 1, wherein the ultrasound generator further includes at least one piezoelectric transducer.

14. The device of claim 13, wherein the piezoelectric transducer is comprised of a piezoelectric stack immediately coupled to a horn-type transducer.

15. The device of claim 14, wherein said horn-type transducer joins with connectors to metal inserts in the mouthpiece.

16. The device in claim 1, wherein the ultrasound generator is operationally responsive to at least one drive signal and is driven about a frequency provided by the drive signal.

17. The device in claim 16, wherein the ultrasound generator further comprises more than one ultrasonic transducer, each transducer being independently operationally responsive to different drive signals.

18. The device in claim 17, wherein at least one first ultrasonic transducer is driven about a first frequency and at least one second ultrasonic transducer is driven about a second frequency that is out of phase with the first frequency.

19. The device in claim 18, wherein the drive signals provide for intermittent operation of the ultrasonic transducers.

20. The device in claim 1, wherein the mouthpiece comprises an acoustic impedance matching device.

21. The device in claim 20, wherein the acoustic impedance matching device is comprised of a material selected from a group consisting of: graphite, metal, and dielectric material.

22. A device for whitening dental tissue, the device comprising:
an ultrasound generator for generating ultrasound, said ultrasound generator comprising:
one or more piezoelectric transducers that oscillate to generate ultrasound; and
one or more electrical connections connected to the one or more piezoelectric transducers,
a mouthpiece attachable to the ultrasonic generator, said mouthpiece comprising:
at least three sides being of a material having an acoustic impedance; and
one or more volumes partially circumscribed by the at least three sides, such that when the mouthpiece is inserted in a mouth of a user one or more teeth are fit into said one or more volumes,
a coupling fluid including a whitening agent and having an acoustic impedance that is about that of the material of the at least three sides of the mouthpiece; said coupling fluid at least partially filling the one or more volumes of the mouthpiece; and
an ultrasonic controller that produces one or more drive signals that are communicated through the one or more electrical connections to the one or more piezoelectric transducers and independently drive the one or more piezoelectric transducers.

23. A method for whitening dental tissue, the method comprising:
inserting a mouthpiece, having one or more volumes partially circumscribed by one or more sides of the mouthpiece, into a mouth of a user such that one or more teeth of the user fit within the said one or more volumes,
filling the volumes partially with a coupling fluid with the coupling fluid including a whitening agent,
controlling the temperature of the coupling fluid within a desired temperature range of 80-90° F.,
controlling the generation of ultrasound; wherein controlling the generation of ultrasound, comprises: providing at least one drive signal; said at least one drive signal comprising at least one frequency,
generating ultrasound with an ultrasound generating means; wherein generating ultrasound comprises: oscillating at least one piezoelectric transducer at said at least one frequency in response to the at least one drive signal; and
coupling the ultrasound through the one or more side of the mouthpiece into the coupling fluid such that ultrasound is directed toward buccal, lingual, and occlusal surfaces of the mouth, wherein coupling the ultrasound comprises: matching an acoustic impedance of the one or more piezoelectric transducers.
